(19) United States
(12) Patent Application Publication
(10) Pub. No.: US 2021/0346500 A1
(43) Pub. Date: Nov. 11, 2021

(54) PHOTOOXIDATIVE INACTIVATION OF PATHOGENS INCLUDING SARS-CoV-2

(71) Applicant: laneg GmbH, Lauenfoerde (DE)

(72) Inventor: Detlef Schikora, Beverungen (DE)

(21) Appl. No.: 17/306,174

(22) Filed: May 3, 2021

Related U.S. Application Data
(60) Provisional application No. 63/020,864, filed on May 6, 2020.

(51) Int. Cl. A61K 41/00; A61L 2/00; A61K 9/00; A61P 31/14; A61K 9/06; C12Q 1/70; A61N 5/06

(52) U.S. Cl. CPC A61K 41/0057; A61N 2005/067; A61L 2/0052; A61L 2/0058; A61K 9/0053; A61P 31/14; A61K 9/0043; A61K 9/06; C12Q 1/70; A61N 5/062; A61N 5/0624; A61N 5/0603; A61L 2202/11; A61N 2005/0651; A61L 2/0076

(57) ABSTRACT

Disclosed in certain embodiments is a method of treating a pathogenic infection comprising (i) contacting a pathogen residing in the oral cavity and/or pharynx of a patient in need thereof with a photosensitizer and (ii) subjecting the photosensitizer contacted pathogen to a light source.

### BACKGROUND

Viruses have been estimated to be the most abundant and diverse biological systems on earth and their size typically ranges from 0.02-0.3 micrometers, though some are larger and can range up to 1 micrometer. Viruses depend on other cells (plant/animal, or bacterial) for their reproduction are classified according to their genome and method of reproduction. They consist of a DNA or RNA (single or double stranded) core, an outer protein cover, and, in some virus classes, lipids.

Coronavirus infection Sars-CoV-2/2019 (COVID-19) is a new pandemic disease. Currently, there are no medications or vaccines available; this has led to dire medical and social consequences and significant morbidity and mortality.

There are also many public health threats from other pathogens such as bacteria (including antibiotic-resistant bacteria), and fungu infections that can give rise serious public health consequences.

There exists a need in the art for new therapies and treatments that can address the public health crisis associated with pathogens such as viruses (e.g. Covid-19), bacetria (e.g., antibiotic resistant bacteria) and fungal infections.

### OBJECTS AND SUMMARY

It is an object of certain embodiments of the invention to provide a method of treating a pathogenic infection comprising (i) contacting a pathogen residing in the oral cavity and/or pharynx of a patient in need thereof with a photosensitizer and (ii) subjecting the photosensitizer contacted pathogen to a light source.

It is an object of further embodiments of the invention to provide the use of a photosensitizer in the treatment of a pathogenic infection comprising (i) contacting a pathogen residing in the oral cavity and/or pharynx of a patient in need thereof with the photosensitizer and (ii) subjecting the photosensitizer contacted pathogen to a light source.

It is an object of other embodiments of the invention to provide the use of a light source in the treatment of a pathogenic infection comprising (i) contacting a pathogen residing in the oral cavity and/or pharynx of a patient in need thereof with a photosensitizer and (ii) subjecting the photosensitizer contacted pathogen to the light source.

It is an object of other embodiments of the invention to provide a kit for the treatment of a pathogenic infection comprising (i) a photosensitizer for contacting a pathogen residing in the oral cavity and/or pharynx of a patient in need thereof and (ii) a light source for subjecting the photosensitizer contacted pathogen to light.

Other objects of the invention are directed to providing a light source that is specifically designated for providing light to the oral cavity and/or pharynx.

Other objects of the invention are directed to providing a photosensitizer composition that is specifically designated for application the oral cavity and/or pharynx for uptake by pathogens (e.g., virus).

Other objects of certain embodiments of the invention include reduction of the pathogenic load (e.g., COVID-19 viral load) in the early stages of the infection; reduction of the pathogenic load in the lung; reduction of inflammation and severe damage in the lung; improving the clinical course of the disease and the reduction of mortality and morbidity and maintaining the capability of forming specific antibodies. These objects are secifically directed to COVID-19 related illness among other pathogenic infections.

### DETAILED DESCRIPTION

In certrain embodiments of the invention, the pathogenic load (e.g., COVID-19 load) is reduced in the initial stages of the disease which helps to lessen the severity of the disease state and minimize subsequnt infection of the lungs, heart, and other organs.

In certain embodimenst of the invention, pathogens such as COVID-19 viruses accumulate the photosensitive molecules or their receptors bind to the photosensitive molecules due to their energetic potential. Photodynamic excitation by an appropriately adapted light source (laser or LED) leads to the formation of reactive singlet oxygen species, which destroy the receptors and/or cellular membrane of the viruses.

While pathogens such as COVID-19 are localized at these sites, they are easily accessible to photooxidative inactivation. When the first symptoms appear and the PCR tests indicate positive results, photodynamic reduction of the pathogenic load can be effected. The treatments and uses of the present invention would then reduce the seeding and pathogenic load to the lower respiratory tract and other organs.

In certain embodiments, the photodynamic process of the present invention does not remove all pathogens (e.g., viruses) which are bound in the oral cavity, throat and nasal cavity. However, may offer an additional advantage, because the reduced pathogenic or viral load stimulates an immune reaction and the formation of protective antibodies, while favoring a mild or moderate course of disease without severe lung dysfunction or damage.

In certain embodiments, the present invention is directed to a method of treating a pathogenic infection comprising (i) contacting a pathogen residing in the oral cavity and/or pharynx of a patient in need thereof with a photosensitizer and (ii) subjecting the photosensitizer contacted pathogen to a light source.

In certain embodiments, the pathogenic infection is a viral infection, a bacterial infection, an antibiotic-resistant bacteria, a fungal infection or a combination thereof.

In certain embodiments, the infection being treated is a systemic infection that is treated by the photosensitizer/light methods of the present invention.

In certain embodiments of the present invention, the photosensitizer is selected from pyrrole derived macrocyclic compounds, porphyrins, chlorins, bacteriochlorins, isobacteriochlorins, phthalocyanines, naphthalocyanines, porphycenes, porphycyanines, pentaphyrins, sapphyrins, benzochlorins, chlorophylls, azaporphyrins, the metabolic porphyrinic precusor 5-amino levulinic acid, synthetic diporphyrins and dichlorins, phenyl-substituted tetraphenyl porphyrins, indium chloride methyl pyropheophorbide, 3,1-meso tetrakis (o-propionamido phenyl) porphyrin, verdins, purpurins, zinc naphthalocyanines, anthracenediones, anthrapyrazoles, aminoanthraquinone, phenoxazine dyes, chlorins, benzoporphyrin derivatives, sulfonated aluminum phthalocyanine, tetrasulfonated derivative, sulfonated aluminum naphthalocyanines, chloroaluminum sulfonated phthalocyanine, phenothiazine derivatives, chalcogenapyrylium dyes, cationic selena and tellurapyrylium derivatives, ring-substituted cationic phthalocyanines, pheophorbide alpha, hydroporphyrins, phthalocyanines, hematoporphyrin, protoporphyrin, uroporphyrin III, coproporphyrin III, protoporphyrin IX, 5-amino levulinic acid, pyrromethane boron difluorides, indocyanine green, zinc phthalocyanine, dihematoporphyrin, benzoporphyrin derivatives, carotenoporphyrins, hematoporphyrin and porphyrin derivatives, rose bengal, bacteriochlorin A, epigallocatechin, epicatechin derivatives, hypocrellin B, urocanic acid, indoleacrylic acid, rhodium complexes, etiobenzochlorins, octaethylbenzochlorins, sulfonated Pc-naphthalocyanine, silicon naphthalocyanines, chloroaluminum sulfonated phthalocyanine, phthalocyanine derivatives, iminium salt benzochlorins, and other iminium salt complexes, DNA-binding fluorochromes, psoralens, acridine compounds, suprofen, tiaprofenic acid, non-steroidal anti-inflammatory drugs, methylpheophorbide-a-(hexyl-ether), and other pheophorbides, furocoumarin hydroperoxides, Victoria blue BO, methylene blue, toluidine blue, porphycene compounds, and combination thereof.

In certain embodiments, the photosensitizer is methylene blue, riboflavin, riboflavin-5-phosphate or a combination thereof. The methylene blue can be derived from, e.g., methylthioninium-chloride dissolved in an aqueous solution such as a sugar or glucose solution.

In certain embodiments, the light source is a laser diode, light emitting diode, infrared and enhanced pulsed light beam or a combinations thereof. In a particular embodiment, the light source utilized in the present invention is a medlouxx device certificated (Germany) according to 93/42/EEC Annex VI and according to EN ISO 13485:2016. Devices disclosed in German Patent No. 10 2016 106804.7 (hereby incorporated by reference) can also be utilized in the present invention.

In certain embodiments, the emitted light is visible light, infrared light or a combination thereof.

In certain embodiments, the virus treated is SARS-CoV-2 (COVID-19), SARS, MERS, swine flu, Zika or a combination thereof.

In certain embodiments, the subjecting of the photosensitizer contacted pathogen to a light source is in the oral cavity. In a particular embodiment, the contacting includes the sublingual region which is highly vascularized.

In certain embodiments, the subjecting of the photosensitizer contacted pathogen to a light source is in the pharynx. In a particular embodiment, the subjecting of the photosensitizer contacted pathogen to a light source is in the nasal cavity, the nasopharynx, the oropharynx or a combination thereof.

In certain embodiments, the treating is initiated within 8 days, 7 days, 6 days, 5 days, 4 days, 3 days, 2 days or 1 day of the onset of infection symptoms.

In certain embodiments, the patient is asymptomatic and the treatment is prophylactic. In other embodiments, the patient is asymptomatic with an infection (e.g., virus infection) and the treatment is initiated to prevent or minimize further onset.

In certain embodiments, the contacting comprises flushing or gargling of the photosensitizer or by application of a viscous formulation (e.g., gel or paste) comprising the photosensitizer.

In certain embodiments, the flushing or gargling can be for any time, such as for about 5 seconds to about 5 minutes.

In certain embodiments, the subjecting of the photosensitizer contacted pathogen to a light source can be for any time, such as for about 30 seconds to about 30 minutes.

In certain embodiments, the light source can emit a wavelength, e.g., of from about 400 nm to about 1000 nm, about 600 nm to about 800 nm, about 650 nm to about 700 nm, or about 660 nm or about 450 nm or about 658 nm.

In certain embodiments, the light source has a power, e.g., of about 10 mW to about 10W about 100 mW to about 500 mW, about 200 mW to about 400 mW or about 240 mW.

In certain embodiments, the contacting and/or the subjecting can be repeated one or more times.

In certain embodiments, the total dosage can be, e.g., about 10 J/cm2 to about 1000 J/cm2, about 50 J/cm2 to about 500 J/cm2, about 60 J/cm2 to about 80 J/cm2, about 300 J/cm2 to about 400 J/cm2, about 72 J/cm2, or about 360 J/cm2 or about 200J/cm2.

In certain embodiments, the treatment further comprises testing the pathogen concentration before treatment, after treatment or a combination thereof. The testing can be, e.g., by polymerase chain reaction.

In certain embodiments, the treating results in a decrease in the pathogen load of the patient. The decreased load can be, e.g., in the oral cavity and/or pharynx of the patient. The decreased load can also be systemic such as in the blood, lungs, heart, gastro-intestinal tract, other organ system, or combination thereof.

### EXAMPLES

We report here, the clinical results of a novel and proprietary research study, using anti-microbial photodynamic treatments to reduce the viral load during the initial stages of the COVID-19 infection with the goal of reducing progression of disease, reducing symptoms, susceptiblity to infectivity, and death while maintaining the ability to mount an immune response

### Materials and Methods

The photosensitizer used was methylene blue as a 1% solution of methylthioninium-chloride dissolved in a 5% glucose solution. (Heltschl GmbH, Germany). The methylene blue solution was applied by flushing and gargling in the oral cavity and throat, and by spraying in the nasal cavity.

The photodynamic excitation was performed using the Medlouxx PDT device, produced by laneg GmbH, Germany (www.medlouxx.com). The device applicator emits 660nm laser radiation with 240 mW power. The infected areas were irradiated for 5 minutes resulting in a dosage of about 72 J/cm2. This procedure: 1 min flushing followed by 5 minutes irradiation, was repeated 5 times, resulting in a total dosage of about 360 J/cm2. Before and immediately after the photodynamic treatments, the viral concentration was determined by PCR tests using the PCR real time testing facility ThermoFisher, QuantStudio 3. The entire treatment procedure is harmless, painless, non-invasive and free of any side effects.

The placebo patients received exactly same treatment procedure except that all placebo patients were treated with covered radiation heads. The patients were not able to distinguish an active versus a placebo treatment due to the filter function of the laser safety goggles.

After 4 weeks patients who had received active treatment were tested with respect to formation of antibodies to SARS CoV-2, using the Euroimmun ELISA test, although this test is not completely specific.

### Results

We have treated 300 patients with the active treatment protocol and 300 patients with the placebo protocol described above. The active treatment group consisted of 164 men and 136 women, with an age range of 35-83 years. All patients signed informed consent prior to the start of the treatment protocol. The main inclusion citeria were fever, typical symptoms and a positive PCR test. The main exclusion criterium was a negative PCR test. Besides fever, we have found a significant variability in the initial presenting symptoms, like cough, loss of smell, loss of taste, headaches, fatigue and others.

We have characterized the course of the disease by the degree and duration of fever which was the most prominent symptom (see Table 1):

TABLE 1
Summary of Symptom Categories

| | | Maximum Fever |
|---|---|---|
| Symptom Category | Maximum Fever | Duration |
| Mild | 37.7° C. (99.9° F.) | 1 Week |
| Moderate | 38.7° C. (101.7° F.) | 2 Weeks |
| Severe (Hospital | 39.2° C. (102.6° F.) | 3 Weeks |
| Admission) | | |
| Severe (ICU/Hospital | 39.5° C. (103.1° F.) | 4-6 Weeks |
| Admission) | | |

Table 2 shows the results regarding the course of the disease based on the symptom categories defined above:

TABLE 2
Summary of Disease Course

| | Active | Active | | |
|---|---|---|---|---|
| | Treatment | Treatment | Placebo | Placebo |
| | Number | Percentage | Number | Percentage |
| Mild | 192 patients | 64% | 102 patients | 34% |
| Moderate | 99 patients | 33% | 141 patients | 47% |
| Severe (Hospital | 6 patients | 2% | 36 patients | 12% |
| Admission) | | | | |
| Severe (Hospital/ | 2 patients | 0.6% | 21 patients | 7% |
| ICU Admission) | | | | |

We have found a significant reduction of severe course of disease (2.6% vs. 19%) and a significant attenuation of disease progression (97% vs. 81%) in the active treatment group of patients. This result is in accordance with a reduced viral load in the oral and nasal cavity and in the throat, measured by PCR test immediately after each 5-stage treatment cycle.

TABLE 3
Summary of Mortality Rate

| Mortality Rate - | Mortality Rate - |
|---|---|
| Active Treatment Group | Placebo Group |
| (300 patients) | (300 patients) |
| 0.7% | 3.3% |

We found a significant reduction in the mortality rate in the active treatment group. The mortality rate in the placebo group was consistent with the average mortality rate in Germany over the same time period.

There were no treatment related adverse events that were noted or suspected.

Discussion

Our results confirm the influence of the viral load on the course of the disease in COVID-19 infections. The percentage of mild disease in the active treatment group was almost double compared to the placebo group.

The percentage of hospital admissions in the placebo group was in accordance with the average data published for Germany in the respective time period. We had 36 patients (12%) with severe disease, requiring hospital admission in the non-treated placebo group and just 6 (2%) patients with severe disease in the photodynamic treated active group.

This result indicates that just the exposure of the viruses to methylene blue by flushing or gargling and spraying does not appear to reduce the viral load; the photodynamic excitation is a necessary process in order to activate a PDI response. The mortality rate differences between the active treatment group and the placebo group, are based upon the assumption that the number of patients in both groups with underlying health conditions such as type 2 diabetes, were quite similar. We cannot make a distinct interpretation of the measured antibody formation rate, because the ELISA tests were not of sufficient specificity to distinguish SARS CoV from SARS CoV-2. We did confirm the presence of antibodies in 96% of the patients of the active treatment group 4 weeks after administering the photodynamic treatments.

### Conclusion

We have investigated the potential of photodynamic treatments in the treatment of COVID-19 infections. We have found and look to further optimize a photodynamic procedure, which is innovative, soon to be uniquely accessible, cost effective and has shown to provide profound clinical efficacy in treating all age groups of those affected by Covid-19. Using methylene blue as a photosensitizer and 660 nm red light for excitation, the viral load in the oral and nasal cavity at the initial stage of the infection can be significantly reduced, leading to significant decreases in morbidity and reduced mortality rates while maintaining the body's ability to mount an immune response and potentially protective immunity in the future. This treatment provides a major breakthrough in the treatment of Covid-19 without any suspected or apparent treatment related adverse events. This is especially relevant for patients who have profound co-morbidities, advanced age, and are at the highest risk as well as potentially asymptomatic carriers who may be continuing to be unwitting participants in the Covid-19 pandemic.

Those of ordinary skill in the art will recognize that many modifications and variations of the present invention may be implemented without departing from the spirit or scope of the invention. Thus, it is intended that the present invention cover the modification and variations of this invention provided they come within the scope of the appended claims and their equivalents.

For simplicity of explanation, the embodiments of the methods of this disclosure are depicted and described as a series of acts. However, acts in accordance with this disclosure can occur in various orders and/or concurrently, and with other acts not presented and described herein. Furthermore, not all illustrated acts may be required to implement the methods in accordance with the disclosed subject matter. In addition, those skilled in the art will understand and appreciate that the methods could alternatively be represented as a series of interrelated states via a state diagram or events.

In the foregoing description, numerous specific details are set forth, such as specific materials, dimensions, processes parameters, etc., to provide a thorough understanding of the present invention. The particular features, structures, materials, or characteristics may be combined in any suitable manner in one or more embodiments. The words “example” or “exemplary” are used herein to mean serving as an example, instance, or illustration. Any aspect or design described herein as “example” or “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “or”. That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. In addition, the articles “a” and “an” as used in this application and the appended claims should generally be construed to mean “one or more” unless specified otherwise or clear from context to be directed to a singular form. Reference throughout this specification to “an embodiment”, “certain embodiments”, or “one embodiment” means that a particular feature, structure, or characteristic described in connection with the embodiment is included in at least one embodiment. Thus, the appearances of the phrase “an embodiment”, “certain embodiments”, or “one embodiment” in various places throughout this specification are not necessarily all referring to the same embodiment.

The disclosure has been described with reference to specific exemplary embodiments thereof. The specification and drawings are, accordingly, to be regarded in an illustrative rather than a restrictive sense. Various modifications of the disclosure in addition to those shown and described herein will become apparent to those skilled in the art and are intended to fall within the scope of the appended claims.

What is claimed is:

1. A method of treating a pathogenic infection comprising (i) contacting a pathogen residing in the oral cavity and/or pharynx of a patient in need thereof with a photosensitizer and (ii) subjecting the photosensitizer contacted pathogen to a light source.

2. The method of claim 1, wherein the pathogenic infection is a viral infection, a bacterial infection, an antibiotic resistant bacteria, an fungal infection or a combination thereof.

3. The method of claim 1, wherein the pathogenic infection is a viral infection.

4. The method of claim 1, wherein the infection is systemic.

5. The method of claim 1, wherein the photosensitizer is methylene blue, riboflavin, riboflavin-5-phosphate or a combination thereof.

6. The method of claim 1, wherein the light source is a laser diode, light emitting diode, infrared and enhanced pulsed light beam or a combinations thereof.

7. The method of claim 1, wherein the emitted light is visible light, infrared light or a combination thereof.

8. The method of claim 1, wherein the virus is SARS-CoV-2, SARS, MERS, swine flu, Zika or a combination thereof.

9. (canceled)

10. The method claim 1, wherein the subjecting of the photosensitizer contacted pathogen to a light source is in the oral cavity.

11. The method of claim 10, wherein the subjecting of the photosensitizer contacted pathogen to a light source is sublingually.

12. The method of claim 1, wherein the subjecting of the photosensitizer contacted pathogen to a light source is in the pharynx.

13. The method of claim 12, wherein the subjecting of the photosensitizer contacted pathogen to a light source is in the nasal cavity, the nasopharynx, the oropharynx or a combination thereof.

14. The method of claim 1 wherein the treating is initiated within 8 days, 7 days, 6 days, 5 days, 4 days, 3 days, 2 days or 1 day of the onset of infection symptoms.

15. The method of claim 1 any of claims 1-13, wherein the patient is asymptomatic with or without proven virus infection.

16. The method of claim 1, wherein the contacting comprises flushing or gargling of the photosensitizer or by application of a viscous formulation (e.g., gel or paste) comprising the photosensitizer.

17. The method of claim 16, wherein the flushing or gargling is for about 5 seconds to about 5 minutes.

18. The method of claim 1 any preceding claim, wherein the subjecting of the photosensitizer contacted pathogen to a light source is for about 30 seconds to about 30 minutes.

19. The method of claim 1, wherein the light source emits light with a wavelength of from about 400 nm to about 1000 nm, about 600 nm to about 800 nm, about 650 nm to about 700 nm, or about 660 nm or about 450 nm or about 658 nm.

20. The method of claim 1, wherein the light source has a power of about 10 mW to about 10W about 100 mW to about 500 mW, about 200 mW to about 400 mW or about 240 mW.

21. The method of claim 1, wherein the contacting and the subjecting is repeated one or more times.

22. (canceled)

23. The method of claim 1, comprising testing the pathogen concentration before treatment, after treatment or a combination thereof.

24. The method of claim 23, wherein the testing is by polymerase chain reaction.

25. (canceled)

26. The method of claim 1, wherein the treating results in a decrease in pathogen load is in the oral cavity and/or pharynx of the patient.

27. (canceled)

28. The method of claim 1, wherein the treating results in a decrease in pathogen load is in the blood, lungs, heart, gastro-intestinal tract, other organ system, or combination thereof.

29. The method of claim 1 wherein the photosensitizer is methylene blue.

30. The method of claim 29, wherein the methylene blue is derived from methylthioninium-chloride dissolved in an aqueous solution.

31. (canceled)

32. (canceled)
